(19) United States
(12) Patent Application Publication
(10) Pub. No.: US 2008/0108113 A1
(43) Pub. Date: May 8, 2008

(54) Methods for the Enrichment of Trehalose Using Alumosilicates

(75) Inventors: Matthias Boy, Langen (DE); Markus Pompejus, Freinsheim (DE); Daniela Klein, Mannheim (DE); Martin Volkert, Ludwigshafen (DE); James Reuben Brown, Mannheim (DE)

Correspondence Address: LAHIVE & COCKFIELD, LLP, ONE POST OFFICE SQUARE, BOSTON, MA, 02109-2127, US

(73) Assignee: BASF AG, Ludwigshafen (DE)

(21) Appl. No.: 10/593,223

(22) PCT Filed: Mar. 18, 2005
(86) PCT No.: PCT/EP05/02936

§ 371 Date: Sep. 17, 2007

(30) Foreign Application Priority Data
Mar. 18, 2004 (DE) 10 2004 013 736.6

(51) Int. Cl. C12P 19/12; C07H 1/06

(52) U.S. Cl. 435/100; 536/127

(57) ABSTRACT

The invention relates to a process for enriching trehalose from solutions, in which the enrichment is performed using an adsorbent, in which the adsorbent is an aluminosilicate. Preferably, the aluminosilicate is a zeolite. The invention further relates to the enrichment and purification of trehalose from fermentation broths, in particular as a coupled product of production by fermentation of other products of value.

The invention hereinafter relates to a process for enriching trehalose from solutions, in which the trehalose is enriched using an adsorbent.

The disaccharide trehalose (α-D-glucopyranosyl-α-D-glucopyranoside) consists of two glucose molecules which are covalently linked to one another via an α,α-1,1 bond. Trehalose, owing to its properties which are of interest in terms of performance is of increasing importance for industry. An important field of application is stabilizing proteins and peptides, for example enzymes and vaccines. A preferred use for trehalose is in the food industry. Trehalose is also used as a substitute for sucrose owing to its reduced sweetness and its properties which preserve taste. In addition, trehalose has a stabilizing action on freezing and drying operations. A further field of application is in the cosmetics sector.

Trehalose is preferably produced enzymatically or by fermentation using suitable microorganisms (Schiraldi, C., et al. (2002). Trehalose Production: Exploiting Novel Approaches. Trends in Biotechnology, vol. 20 (10), pages 420-425). Frequently, trehalose is also formed as a byproduct in fermentations which serve for the production of other substances (Hull, S. R., Gray, J. S. S., et al. (1995). Trehalose as a Common Industrial Fermentation Byproduct. Carbohydrate Research, vol. 266, pages 147-152). In particular in the case of fermentations, other than with chemical syntheses, highly contaminated solutions are formed which can contain, for example, cells, proteins, lipids, or other sugars.

The trehalose must therefore be enriched from such highly contaminated solutions, and, depending on the intended use, be further purified.

In the prior art, various enrichment and purification processes for trehalose are known.

U.S. Pat. No. 5,759,610 describes a process for purifying trehalose from cultures of microorganisms comprising the steps filtration and centrifugation, treatment with activated carbon, deionization, purification with ion exchangers, concentration to form syrupy products, further purification by column chromatography techniques such as ion-exchange column chromatography, activated carbon chromatography and silica gel column chromatography, and also precipitation with organic solvents such as alcohol and acetone and filtration through suitable membranes, and fermentation by yeast or alkaline treatment in order to remove or break down any remaining saccharides. For further purification, cooling crystallization or spray drying, for example, are proposed. Adsorption of trehalose to an adsorbent is not performed.

JP 07000190 (Tradashi, W., et al.) describes the isolation of trehalose from solid residues of brewery fermentations. The residue is extracted with alcohol and/or treated with ultrasound to extract the trehalose from the residue. Furthermore, the enzyme trehalase present in the residue is inactivated by heat treatment. Purification is performed, inter alia, via ion-exchange columns and one activated-carbon column. The trehalose is not adsorbed to the columns in this process.

U.S. Pat. No. 5,441,644 describes a process in which trehalose is purified from a fermentation broth. In the process, inter alia, an ultrafiltration and decolorization using activated carbon are performed. The trehalose is not adsorbed to the activated carbon in the process.

A disadvantage of said processes appears to be that the respective adsorbents are used only for the adsorption of the unwanted foreign matter, but do not adsorb the trehalose itself. Since the extraction and purification steps must be adapted to the differing foreign matter, they are complicated and only applied with difficulty on an industrial scale. In particular, this applies to purification from fermentation broths in which the trehalose content is usually less than 15% of the dry weight (Schiraldi et al. (2002), Trehalose Production: Exploiting Novel Approaches. Trends in Biotechnology, vol. 20 (10), page 421).

According to another process, trehalose was purified as a byproduct of a fermentation by sequential chromatography on activated carbon and Bio-Gel P-2 (Hull, S. R., Gray, J. S. S., et al. (1995). Trehalose as a Common Industrial Fermentation Byproduct. Carbohydrate Research, vol. 266, pages 147-152). The process, however, is only a detection method, not a process which is suitable for application on an industrial scale.

U.S. Pat. No. 5,441,644 mentions, in addition to the above described process, a further process of the prior art in which a trehalose-containing acetonitrile solution is subjected to a silica-gel chromatography. The publication mentions that these chromatographic processes are unsuitable, however, for trehalose enrichment or trehalose purification on an industrial scale.

Buttersack et al. (Specific Adsorption from Aqueous Phase on Apolar Zeolites, Progress in Zeolite and Microporous Materials, vol. 105, pp. 1723-1730, 1997) describe the binding of certain mono- and disaccharides to selected FAU, PEA and MFI zeolites. For individual disaccharides, highly differing adsorption properties were found. Trehalose was not studied.

In a further work, Buttersack et al. describe the binding of disaccharides to differing Y zeolites and dealuminized Y zeolites (Buttersack et al. (1994). Adsorption of Glucose and Fructose containing Disaccharides on Different Faujasites. Studies in Surface Science and Catalysis, vol. 84, pp. 1363-1371). They stress the importance of the fructose radical in the disaccharides studied for adsorption to the zeolites. Trehalose was not studied and also does not have a fructose radical.

A disadvantage of the previous adsorbents is that they have very general adsorption properties and cannot be adjusted individually for the respective process.

Therefore there is a requirement for processes for enriching trehalose from solutions using better adsorbents, in particular for adsorbents which may be tailored to the respective process. It is an object of the present invention, therefore, to provide such a process, in particular for use in chromatographic processes. It is a further object of the present invention to provide a process which makes it possible to enrich trehalose from fermentation broths, in particular from lysine production fermentation broths.

We have found that this object is achieved starting from the known process for enriching trehalose from solutions using an adsorbent. A feature of the inventive process is that the adsorbent is an aluminosilicate.

Compared with the adsorbents used according to the prior art (for example activated carbons and ion exchangers), aluminosilicates, in particular zeolites, offer the advantage that a greater number of variants can be prepared, and as a result the adsorbent can be tailored better to the separation problem.

Trehalose can be produced by a multiplicity of known processes. Traditionally, trehalose is produced by fermentation processes, with, in the meantime, enzymatic production processes also having become established (Schiraldi, C., et al. (2002) Trehalose Production: Exploiting Novel Approaches. Trend in Biotechnology, vol. 20 (10), pp. 420-425). In microorganisms, 3 main enzymatic routes have been discovered for trehalose synthesis: (1) a phosphorylase system in fungi and yeast, (2) a glucosyltransferase-hydrolase system in mesophilic and extremophilic bacteria and (3) a trehalose-synthase catalyzed transglycosilation of maltose to trehalose (for example JP 09098779, KR99029104).

The term enrichment is known to those skilled in the art. In accordance with the present invention, the term enrichment relates in particular to increasing the proportion of trehalose in relation to unwanted foreign matter. Typically, this proportion of trehalose corresponds to the dry weight of the product.

In the preferred embodiment, the term enrichment also relates to the purification of trehalose. The term purification is known to those skilled in the art. In the present context it is in particular a purpose of purification to achieve a trehalose purity in which the trehalose is essentially free from other substances. In particular, this means trehalose in crystalline form.

An enrichment or purification process is only economically expedient if the yield is satisfactory. Therefore, it is a further purpose of the present process to achieve not only a high enrichment but also a high yield.

Regarding the solution, there are no special restrictions with respect to the solvents, those which can be used are, for example, water or acetonitrile. Preferably, the solution is an aqueous solution.

An adsorbent within the meaning of the present invention is a solid or gel-like substance on the surface of which the adsorption of another substance takes place. The term surface here relates also to the internal surface of a three-dimensional matrix, for example the internal surfaces of the three-dimensional framework of a zeolite.

Examples of adsorbents within the meaning of the present invention are silica gel, activated carbon and aluminosilicates.

Aluminosilicates are known to those skilled in the art. The term aluminosilicates comprises, for example, acid-activated bentonites (bleaching earths) and zeolites. Acid-activated bentonites (bleaching earths) are bentonites, the smectites of which (swellable or clay minerals) have been partially dissolved by acid treatment and which thus have a high surface area and a large micropore volume. Bentonites are clays which have been formed by the weathering of volcanic ash (tufa) and consist of the minerals montmorillonite and beidellite (the smectite mineral group).

Particularly preferred aluminosilicates in the context of the present invention are zeolites. In this context, those zeolites which do not contain aluminum can also come under the invention.

Zeolites are a widely distributed group of crystalline silicates, more precisely of water-containing alkali metal or alkaline earth metal aluminosilicates of the general formula M₂/_{z}O.Al₂O₃.x SiO₂.y H₂O, where M=monovalent or polyvalent metal (usually an alkali metal or an alkaline earth metal cardion) H or NH₄ etc., z=the valency of the cation, x=from 1.8 to about 12 and y=from 0 to about 8. The stoichiometric ratio of SiO₂ to Al₂O₃ (modulus) is as important parameter of zeolites.

The crystal lattice of zeolites is built up from SiO₄ and AlO₄ tetrahedra which are linked via oxygen bridges. This produces an arrangement in space of equally constructed (adsorption) cavities which are accessible via channels or pore openings, which are of equal sizes among one another. Crystal lattices of this type are able to act as a sieve which admits molecules having a smaller cross section than the pore openings into the cavities of the lattice, while larger molecules cannot penetrate. Zeolites are therefore also termed molecular sieves. Electrostatic interactions, hydrogen bonding and other intermolecular forces also play a role in the adsorption. Many chemical and physical properties of zeolites are dependent of the Al content.

The term zeolites according to the present invention relates not only to natural but also to synthetic zeolites.

The naturally occurring zeolites are formed by hydrothermal conversion from volcanic glasses or tufa-containing deposits. According to their crystal lattices, the natural zeolites may be classified into fibrous zeolites (for example mordenite, MOR), leaf zeolites and the cubic zeolites (for example faujasite, FAU, and offretite, OFF). The differing zeolites are usually given three-letter codes (for example MOR, FAU, OFF).

To prepare synthetic zeolites, the starting materials used are SiO₂-containing (for example waterglasses, silica fillers, silica sols) and Al₂O₃-containing (for example aluminum hydroxides, aluminates, kaolins) substances which, together with alkali metal hydroxides (usually NaOH) are converted to the crystalline zeolites at temperatures above 50° in the aqueous phase.

For industrial use as adsorbents, synthetic zeolites can be subjected to further modifications. Preferably, the zeolite should have a pore size of at least 7 Å. Pore size and polarity of zeolites have an influence on the distribution weight, for example of different sugars, which gives, for example, the separation property in a chromatographic application. Low-aluminum zeolites are generally polar and thus of priority for the adsorption of sugars.

As already described, zeolites can readily be tailored to a separation problem. The primary preparation can affect the pore size, and the polarity can then be varied via a post-treatment by reducing the aluminum content.

Preferred zeolites according to the present invention are FAU, BEA and OFF. Properties which are respectively advantageous of different zeolites in the context of the present invention can be seen in example 1. Particular preference is given to OFF.

Enrichment using the aluminosilicate can take place in principle in two different ways. The aluminosilicate can either adsorb the unwanted foreign matter so that the trehalose remains in solution, or it can adsorb the trehalose so that the unwanted foreign matter remains in solution. In both cases it is preferable if the adsorption takes place as selectively as possible.

As adsorber, use can be made of fixed-bed, moving-bed and fluidized-bed adsorbers. The adsorption can be carried out batchwise or continuously.

In the embodiment in which trehalose is adsorbed to the aluminosilicate, a number of advantages arise. The number of the required work-up steps for isolating trehalose is reduced by selective enrichment of trehalose (in contrast to previous processes for isolating trehalose in which the frequently highly varied unwanted foreign matter has to be removed step by step). The number of byproduct/waste streams is reduced compared with the stepwise removal of the unwanted foreign matter. Trehalose, owing to selective adsorption, is present at high purity even after a primary enrichment step using the aluminosilicate. Owing to the decreased number of workup steps and the reduced number of byproduct/waste streams, the production costs are reduced. In addition, trehalose of comparatively low concentration can be cost-effectively enriched by selective enrichment.

Preferred aluminosilicates in this embodiment are therefore aluminosilicates, in particular zeolites, to which trehalose adsorbs, preferably bind with high selectivity compared with unwanted foreign matter present in the solution.

After the trehalose is adsorbed to the aluminosilicate, as a further step, the trehalose can be eluted from the aluminosilicate. It is eluted, for example, by eluting with methanol, ethanol, water, hot water (50-100° C.), hot methanol (50-65° C.), hot ethanol (50-80° C.) or other suitable eluents, for example methylene chloride, acetonitrile, NMP (N-methyl-2-pyrrolidone), DMSO (dimethyl sulfoxide), short-chain ketones or short-chain ethers. Short-chain in this context means a chain length of up to C10, preferably up to C6, particularly preferably up to C4.

A further embodiment of the invention relates to a process for enriching trehalose in which the adsorbent is used in the context of a chromatographic separation. In chromatographic processes, the trehalose can be separated via the different running time behavior compared with other substances present in the solution. This produces fractions with eluates which contain the trehalose.

Within the meaning of the present invention, the term chromatography comprises all known and suitable chromatographic separation processes, for example fixed-bed chromatography, moving-bed chromatography and simulated moving-bed chromatography. The chromatography can be carried out batchwise or continuously. Continuous chromatography can be carried out, for example, using a Continuous Rotating Annular Chromatograph (CRAC), a True Moving-Bed Chromatograph (TMBC) or a Simulated Moving-Bed Chromatograph (SMB).

From the trehalose-containing eluate, a further enrichment or purification can be performed by means of further processes which are suitable and known to those skilled in the art.

For example, further enrichment or purification of trehalose can take place by precipitation. In this step, either wanted materials of value or unwanted foreign matter can be precipitated out. The precipitation can be initiated, inter alia, by adding a further solvent, adding salt or varying the temperature. The resultant precipitate of solids can be separated off by processes known to those skilled in the art.

For example, solids can be separated off by filtration, such as pressure and vacuum filtration. It is also possible to use cake filtration, depth filtration and cross-flow filtration. Preference is given to cross-flow filtration. Particular preference is given here to microfiltration for separating off solids >0.1 μm.

A further possibility for separating off solids is sedimentation and/or centrifugation. For centrifugation, various types of constructions can be used, for example tube and basket centrifuges, especially pusher, inverting filter centrifuges and disk separators.

As a further enrichment or purification step, treatment with activated carbon or with ion exchangers (anion exchangers and/or cation exchangers) can be carried out. Process steps of this type are known from the prior art (see, for example, U.S. Pat. No. 5,441,644, U.S. Pat. No. 5,858,735 and EP 0 555 540 A1).

Further possibilities for enrichment, in particular for purification, are the use of microfiltration and ultrafiltration (for example as cake, depth and cross-flow filtration techniques) and reverse osmosis. In this case, inter alia, microporous, homogeneous, asymmetric and electrically charged membranes can be used, which are produced by known processes. Typical materials for membranes are cellulose esters, nylon, poly(vinyl chloride), acrylonitrile, polypropylene, polycarbonate and ceramics.

The membranes can be used, for example, as a plate module, spiral module, tube bundle and hollow-fiber module. In addition, the use of liquid membranes is possible. The trehalose can be not only enriched on the feed side and removed via the retentate stream, but also depleted on the feed side and removed via the filtrate/permeate stream.

For further enrichment of trehalose, in particular for purification and final processing, various methods known to those skilled in the art can be used. A preferred process here is crystallization. Crystallization can be achieved, for example, by cooling, evaporation, vacuum crystallization (adiabatic cooling), reaction crystallization and salting out. The crystallization can, for example, in stirred and unstirred tanks, in the direct-contact process, in evaporative crystallizers, in vacuum crystallizers batchwise or continuously, for example in forced-circulation crystallizers (Swenson forced-circulation crystallizers) or fluidized-bed crystallizers (Oslo type). Fractional crystallization is also possible.

The crystallization of trehalose is familiar in principle to those skilled in the art and has been extensively described, including crystallization from aqueous solutions (see also columns 4 and 5 in U.S. Pat. No. 5,441,644). For instance, crystallization can be achieved, for example, by previous ultrafiltration.

A particularly typical method for crystallizing trehalose is cooling crystallization from suitable solvents, for example ethanol, methanol, water, methylene chloride, acetonitrile, NMP, DMSO, short-chain ketones or short-chain ethers. Short-chain in this context denotes a chain length of up to C10, preferably up to C6, particularly preferably up to C4. Another crystallization method is precipitation crystallization. In this method the trehalose is present, for example in water, and is then precipitated by adding a solvent of lower solubility, for example a short-chain alcohol or a short-chain ketone. Short-chain in this context denotes a chain length of up to C10, preferably up to C6, particularly preferably up to C4.

The crystallization can be accelerated by adding small amounts of trehalose crystals, the trehalose crystals acting as crystallization seeds.

Other processes exist for the further enrichment of trehalose; in particular, for purification and final processing, there is drying. There exist processes for convection drying, for example drying ovens, tunnel driers, belt driers, disk driers, jet driers, fluidized-bed driers, aerated and rotating drum driers, and spray drying. A preferred process in the context of the present invention is spray drying. Further processes utilize contact drying, for example blade driers. Likewise, heat radiation (infrared) and also dielectric energy (microwaves) can be used for drying. A further field is vacuum or freeze drying. Condensation is also possible, that is to say drying which leads to enrichment, but not necessarily to dryness.

A further process for the further enrichment of trehalose, in particular for purification and final processing, is nanofiltration. In this process the trehalose is wholly or partly retained on the retentate side and thus enriched.

It is obvious to those skilled in the art that said further enrichment steps can be carried out not only before but also after the inventive treatment with the aluminosilicate.

In a further embodiment, the present invention relates to a process for enriching trehalose from solutions which originate from the enzymatic synthesis of trehalose. Enzymatic trehalose synthesis is known to those skilled in the art (see, for example, Schiraldi et al. (2002), Trehalose Production: Exploiting Novel Approaches. Trends in Biotechnology, vol. 20 (10), pages 421-425, and also U.S. Pat. No. 5,919,668 and EP 0 990 704 A2).

In a further embodiment the solutions are fermentation broths.

Fermentation broths within the meaning of the present invention are produced in the culture of eukaryotic and prokaryotic cells, in particular microorganisms (for example bacteria, yeasts or other fungi).

Preferred microorganisms in the synthesis of trehalose are Saccharomyces spec., in particular Saccharomyces cerevisiae; Bacillus spec.; Candida spec., in particular Candida fermentii; Escherichia coli; Corynebacterium spec., in particular Corynebacterium glutamicum, Corynebacterium acetoacidofirum (for example ATCC 13870), Corynebacterium lilium (for example ATCC 15990) and Corynebacterium melaseccola (for example ATCC 17965); Pseudomonas spec.; Nocardia spec.; Brevibacterium spec., in particular Brevibacterium lactofermentum (for example ATCC 13869), Brevibacterium flavum (for example ATCC 14067), and Brevibacterium divaricatium (for example ATCC 21642); Arthrobacter spec., in particular Arthrobacter sulfureis (for example ATCC 15170), Arthrobacter citoreus (for example ATCC 11624); Aspergillus spec.; Streptomyces spec.; Microbacterium spec., in particular Mikrobacterium ammoniaphylum (for example ATCC 15354); Pichia spec.; Filobasidium spec., in particular Filobasidium floriforme.

Further suitable microorganisms are known to those skilled in the art, see, for example, Miyazaki, J.-I., et al. (1996)., Trehalose acumulation by a basidiomycotinous yeast, Filobasidium floriforme. Journal of Fermentation and Bioengineering, vol. 81 (4), pages 315-319.

Variants of these strains which are derived by mutation or genetic modification, or which have an increased trehalose synthesis ability, can also be used in the context of the present invention.

The microorganisms can also be cultured with the addition of suitable antibiotics, for example for inducing trehalose synthesis by adding a β-lactam ring antibiotic.

The fermentation broth comprises in this case firstly not only the cells, but also the culture medium. Depending on the type of fermentation, a significant part of the trehalose can accumulate intracellularly. In this case it is expedient to digest cells used and to extract the trehalose using suitable methods. Suitable methods, for example ultrasound treatment, treatment with detergents, alkaline lysis and/or extraction with alcohol or trichloroacetic acid are known to those skilled in the art (JP 07 000 190, U.S. Pat. No. 5,441,644).

In the fermentation broth there are generally considerable amounts of solids which should preferably first be separated off.

The term solids also comprises in the present context cells and cellular constituents such as nucleic acids and proteins. To separate off solids, in particular cellular constituents, it is advantageous first to agglomerate these. This can be performed with any suitable processes, however in this case a breakdown of the trehalose (for example by hydrolysis) should largely be avoided. Suitable methods comprise, for example, alkali treatment, for example Ca(OH)₂ treatment, or heating. Advantageously, in this case, enzymes having trehalase activity which are possibly present are also inactivated.

The solids can then be separated off by processes known to those skilled in the art. Examples of such processes have already been mentioned above.

The present process is also suitable for enriching trehalose from solutions, in particular fermentation broths, in which trehalose is present at low concentrations, in particular less than 15 percent by weight, measured on the dry weight of the fermentation broth.

Typically, the trehalose concentration is from 3 to 8% by weight, measured on the dry weight of the fermentation broth. After separating off another product of value, for example lysine, the mass fraction of trehalose can increase to 10-20% by weight, measured on the dry weight of the remaining fermentation broth. If separation of the biomass as insoluble constituents is also used at the starting point, the trehalose concentration is then 20-40% by weight, measured on the dry weight of the fermentation broth.

Therefore, a further embodiment of the invention is also a process for enriching trehalose from fermentation broths in which trehalose is present at a concentration less than 15 percent by weight, measured on the dry weight of the fermentation broth.

In many fermentations, a plurality of products of value are produced. Frequently, trehalose is also produced as a further product of value. A problem is then that enrichment or purification processes for substances produced by fermentation is specifically adapted to the respective product of value (for example purification via ion-exchange chromatography in the case of amino acids or organic acids). After the enrichment of the first product of value, other products of value such as trehalose are actually present in an environment which hinders the enrichment of the further products of value. An example is high ion concentrations after eluting amino acids from ion exchange matrices). This is particularly problematic in the case of trehalose, since trehalose does not have special chemical properties (for example low solubility in aqueous solutions or electrical charge) which are suitable for a simple enrichment. Therefore, the trehalose is frequently disposed of together with the waste stream from the fermentation.

It is therefore a further object of the present invention to work up trehalose as a further product of value from fermentation broths from which a first product of value has been or is worked up in advance or subsequently.

In a further embodiment the present invention therefore relates to a process for enriching trehalose from a further product of value from fermentation broths from which at least one first product of value has been or is obtained, comprising the steps of separating off solids and enriching the trehalose using an adsorbent, wherein the adsorbent is an aluminosilicate.

The present process is distinguished in that it is particularly tolerant toward the properties of the solution in which the trehalose is present. Therefore, the inventive process can also be used when the trehalose is present in an environment which would usually hinder the enrichment.

Conversely, the solution in which the trehalose is present is treated particularly gently by the present process, so that a further product of value can be obtained even after the enrichment of the trehalose.

Therefore, the trehalose can be obtained before, after or at the same time as the first product of value.

Products of value within the meaning of the present invention comprise, for example, organic acids, proteinogenic and nonproteinogenic amino acids, nucleotides and nucleosides, lipids and fatty acids, diols, carbohydrates, aromatic compounds, vitamins and co-factors, storage substances, for example PHA (polyhydroxyalkanoates) or PHB (polyhydroxybutyrates), and also proteins and peptides (for example enzymes).

A preferred first product of value according to the present invention is the amino acid lysine.

In the exemplary embodiments, further processes are shown which are suitable for purifying trehalose from fermentation broths from which another product of value was obtained in advance.

The drawings and examples serve for more detailed illustration of the invention.

The accompanying drawings show, in

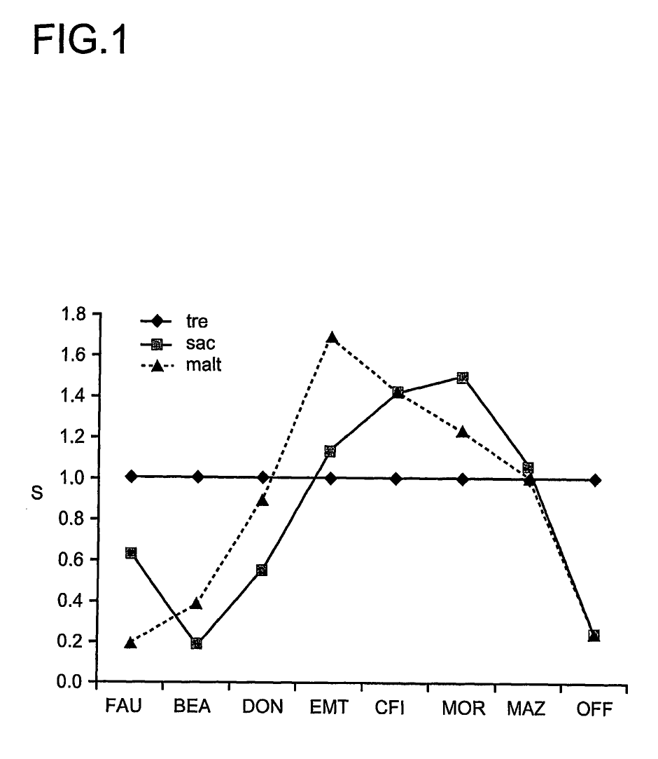

FIG. 1 the selectivity (s) of zeolites for sucrose (sac) and maltose (malt) relative to trehalose (tre).

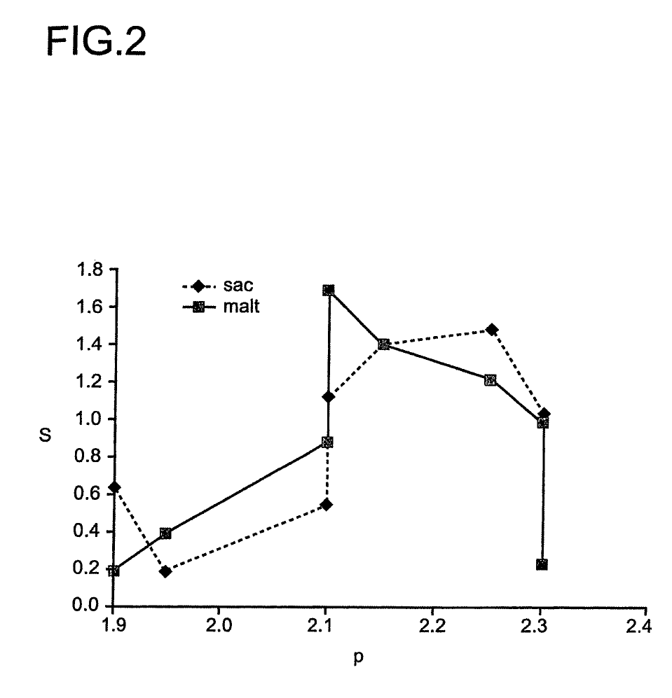

FIG. 2 the selectivity (s) for sucrose (sac) and maltose (malt) relative to trehalose in relation to pore size (p) of selected zeolites.

Determination of pore size: space-filling atom-centered spheres are used to represent the van der Waals volumes for the atoms, the radii of the spheres corresponding to the van der Waals radii, as are defined in the MSI Program Materials Studio. An expansion factor of 0.9 is applied to the van der Waals radii of the atoms in the zeolite pore and a helium atom is then placed in the center of the pore. The expansion factor for the helium van der Waals radius is optimized by hand until the expanded space-filling volume of the helium atom comes into contact with the space-filling volumes of the zeolite pore. This helium expansion factor is used as expansion factor of the pore (pore size).

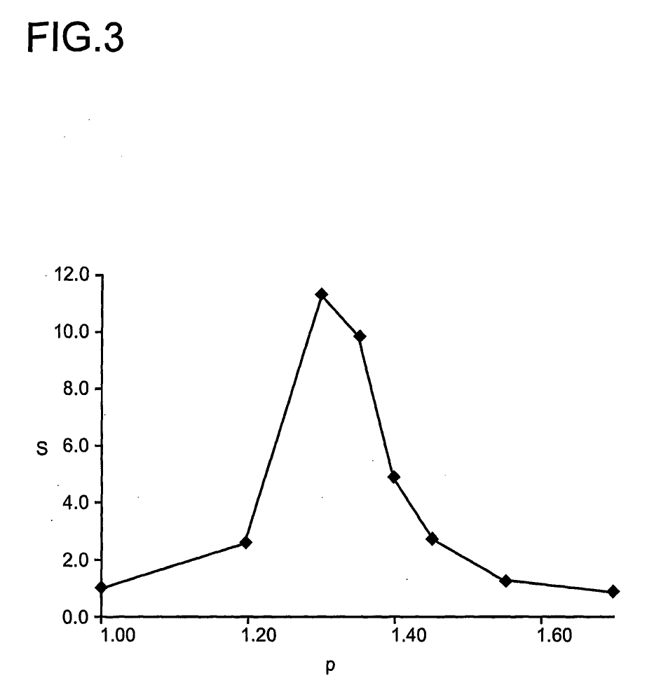

FIG. 3 The selectivity (s) for hydrocarbons in relation to the pore size (p) of selected zeolites.

### EXAMPLE 1

To compare the diffusion of sugars in various zeolites quantitatively, theoretical calculations are made. In these, conventional dynamic molecular simulations are carried out along a diffusion coordinate. The diffusion coordinate is determined by a small driving force which is applied along the axis of the widest pore or the widest channel. This simulates the effect of a concentration gradient.

A study is first made as to whether the simulation yields qualitatively correct results. For this purpose, the calculated diffusion times for maltose and sucrose in FAU and BEA are compared with experimental measurements. According to the calculations, maltose diffuses markedly slower than trehalose and sucrose through FAU (see table 1). This is in agreement with the experimental data which show that maltose has a markedly lower adsorption capacity than sucrose.

For BEA it is calculated that sucrose, in the context of the time scale used, does not migrate at all through the zeolite (see table 2). This effect (no adsorption) is a general characteristic of other 1-2 Fru disaccharides which were measured experimentally. From these results for BEA and FAU, it is concluded that the calculation yields qualitatively correct predictions for the relative “solubility” of maltose and sucrose in FAU and BEA.

First a list of candidates for suitable zeolites for separating trehalose, maltose and sucrose is formed (table 1).

TABLE 1

| | | Calculated |
|---|---|---|
| | Actual composition | composition |
| DON | [Si₆₄O₁₂₈]•2(Cp*)2CoF_{0.75}(OH)_{0.25}) | Si₆₄O₁₂₈ |
| EMT | Na₂₁(18-crown-6)ₙ[Al₂₁Si₇₅O₁₉₂] | Al₂₁Si₇₅O₁₉₂ |
| CFI | [Si₃₂O₆₄] | Si₃₂O₆₄ |
| MOR | Na₈[Al₈Si₄₀O₉₆]•24H₂O | Si₄₀O₉₆ |
| MAZ | (Na₂,K₂,Ca,Mg)₅[Al₁₀Si₂₆O₇₂]•28H₂O | Al₁₀Si₂₆O₇₂ |
| OFF | (Ca,Mg)_{1.5}K[Al₄Si₁₄O₃₆]•14H₂O | Si₁₈O₃₆ |
| FAU | (Na₂,Ca,Mg)₂₉[Al₅₈Si₁₃₄O₃₈₄]•240H₂O | Al₉₆Si₉₆O₃₈₄ |
| BEA | Naₙ[AlₙSi₆₄₋ₙO₁₂₈] | Si₆₄O₁₂₈ |

Dynamic molecular simulations are then carried out using these zeolites for all 3 sugars. In this manner the relative selectivity of the sugars with regard to diffusion through the corresponding channels can be calculated.

The dynamic molecular force field simulations are carried out in a microcanonical ensemble at 298 K. The relative times are measured for molecules which are driven through a pore in the zeolite structure by electrostatic force. The force is generated by the means that the coordinates of the charged helium atom are fixed on the opposite side of the pore of the molecule, the molecule then being uniformly charged with a corresponding countercharge on each atom. For example, the 5 atoms of trehalose which are closest to the helium are each assigned a charge of −0.3 q, while the helium atom has a charge of +1.5 q. The remaining atoms in the system are uncharged. The selectivity in FIG. 1 is calculated according to the formula below:

$$Selectivity = \frac{{t}_{trehalose}}{{t}_{sugar}} where , {t}_{sugar} = 8000 ps , \\ when {t}_{sugar} is > 8000 ps$$

The calculated diffusion times for the sugars are listed in table 2.

TABLE 2

| | Trehalose | Sucrose | Maltose |
|---|---|---|---|
| FAU | 1500 | 2400 | 8000 |
| BEA | 1500 | 8000 | 3900 |
| DON | 2400 | 4400 | 2700 |
| EMT | 5100 | 4500 | 3000 |
| CFI | 1700 | 1200 | 1200 |
| MOR | 2400 | 1600 | 1950 |
| MAZ | 1800 | 1700 | 1800 |
| OFF | 2000 | 8000 | 8000 |

A graphical representation of the selectivity is shown in FIG. 1. From FIG. 1 it becomes clear that the individual zeolites have differing capacities for separating trehalose from a mixture of sugars. The most versatile appears to be OFF (offretite) which does not contain aluminum and prefers trehalose markedly compared with the other two sugars. FAU and BEA likewise show a high relative selectivity for trehalose, but also show a certain selectivity for sucrose and maltose.

### EXAMPLE 2

Enrichment of Trehalose by Precipitation With Calcium Hydroxide, Centrifugation of Subsequent Activated Carbon Treatment and Drying of the Residue

1 l of lysine fermentation broth is admixed with 250 g of solid calcium hydroxide after the lysine has been separated off on an ion exchanger. After the suspension has been stirred for 4 hours, the suspension is centrifuged in a laboratory centrifuge at 3000 g for 10 min. As a result of this procedure, 800 ml of a yellowish supernatant are obtained from the deep-brown fermentation broth, which supernatant comprises 7.6 g of the 8 g of trehalose originally used. For further purification of this supernatant, 400 g of pulverized activated carbon are added. After incubation for 12 hours at room temperature, the activated carbon is separated off via a fluted filter. 650 ml of a slightly yellowish filtrate are obtained, which contains in total 6.3 g of trehalose. Finally, the filtrate is freeze-dried. The remaining residue of 9.7 g has a trehalose content of 64.9% by weight.

### EXAMPLE 3

Enrichment of Trehalose by Precipitation With Calcium Hydroxide, Filtration, Subsequent Activated Carbon Treatment and Drying of the Residue

In contrast to example 2, after the calcium hydroxide precipitation, the solids formed are separated off by filtration. This produces 730 ml of a yellowish filtrate. The further procedure is performed in a similar manner to example 2, as a result of which 8.7 g of dry residue having a trehalose content of 66.2% by weight can be obtained.

### EXAMPLE 4

Enrichment of Trehalose by Thermally Induced Precipitation, Cross-Flow Filtration, Subsequent Activated Carbon Treatment and Drying of the Residue

### EXAMPLE 5

Enrichment of Trehalose by Precipitation With Calcium Hydroxide, Centrifugation of Subsequent Activated Carbon Treatment and Drying of the Residue (Broth From New Workup)

1 l of lysine fermentation broth, after the lysine has been separated off on an ion exchanger (trehalose content: 11 g/l), is admixed with 100 g of solid calcium hydroxide. After the suspension has been stirred for 4 hours, the suspension is centrifuged in a laboratory centrifuge at 3000 g for 10 min. 20 g of activated carbon are added to the resultant 800 ml of a dark-brown supernatant and the mixture is incubated at RT for 19 h. The activated carbon is separated off by filtration. The filtrate contains 8.9 g of trehalose. By concentration in vacuo, 72.6 g of a dark-brown sticky residue having a trehalose content of 10.4% by weight are obtained.

### EXAMPLE 6

Enrichment of Trehalose by Adsorption to Activated Carbon and Desorption With Methanol

100 ml of a trehalose-containing fermentation broth (content 9.76 gf/l) are shaken with 10 g of activated carbon (CPG 12×40) at RT for 16 h. After the mixture is filtered off with suction via a slotted screen suction filter, the activated carbon is shaken with 100 ml of methanol at RT for 60 h. After renewed filtration, the filtrate is concentrated to dryness on a rotary evaporator. The brown residue of 1.1 g contains 300 mg of trehalose (27% by weight).

### EXAMPLE 7

Enrichment of Trehalose by Adsorption to Activated Carbon and Desorption With Ethanol Under Cooling Crystallization

300 ml of a trehalose solution (content 9.25 g/l) are shaken with 20 g of activated carbon at RT for 18 h. After the mixture is filtered off by suction via a slotted screen suction filter, the activated carbon is admixed with 300 ml of ethanol and stirred under reflux for 15 h. The activated carbon is filtered off hot and the filtrate is cooled to 0-5° C., with the trehalose crystallizing out. After filtering the mixture off with suction, 1.3 g of trehalose are obtained as light-gray crystals, the filtrate is concentrated to dryness on a rotary evaporator and contains 0.1 g of trehalose as white crystals.

The activated carbon, after the filtration, is shaken with 300 ml of MeOH at RT for 16 h, filtered and off the filtrate is concentrated on a rotary evaporator, as a result a further 0.5 g of trehalose is obtained as virtually white crystals.

### EXAMPLE 8

Enrichment of Trehalose by Adsorption to Silica Gel and Desorption With Methanol

100 ml of a trehalose-containing fermentation broth (content 14 g/l) are shaken with 10 g of silica gel (MR482) at RT for 19 h. After the mixture is filtered off with suction via a glass suction filter, the silica gel is shaken with 100 ml of methanol at RT for 16 h. After repeated filtration, the filtrate is concentrated to dryness on a rotary evaporator. The brown residue of 1.5 g contains 110 mg of trehalose (7% by weight).

1. A process method for enriching trehalose in a solution, comprising exposing the solution to an adsorbent comprising a zeolite, thereby enriching trehalose in said solution.

2. (canceled)

3. The method of claim 1, wherein the trehalose is adsorbed to the zeolite.

4. The method of claim 1, wherein the zeolite is selected from the group consisting of FAU, BEA, DON, EMT, CFI, MOR, MAZ and OFF.

5. The method of claim 1, wherein the adsorbent is used during chromatographic separation.

6. The method of claim 1, wherein the solution originates from an enzymatic trehalose synthesis.

7. A method for enriching trehalose in a fermentation broth, comprising separating off solids from the broth and exposing the broth to an adsorbent comprising a zeolite, thereby enriching trehalose in said fermentation broth.

8. (canceled)

9. The method of claim 7, further comprising separating off from the fermentation broth at least one additional product.

10. The method of claim 7, wherein the fermentation broth originates from the fermentation of at least one microorganism selected from the group consisting of Saccharomyces spec., Candida spec., Escherichia coli, Corynebacterium spec., Corynebacterium glutamicum, Pseudomonas spec. Nocardia spec., Brevibacterium spec., Arthrobacter spec., Streptomyces spec., Microbacterium spec., Aspergillus spec., Bacillus spec., Pichia spec. and Filobasidium spec.

11. The method of claim 7, wherein the trehalose is present in the fermentation broth at a concentration of less than 15 percent by weight measured on the dry weight of the fermentation broth.

12. The method of claim 7, further comprising purifying the trehalose by a method selected from the group consisting of activated carbon treatment, ultrafiltration microfiltration, reverse osmosis and ion-exchange treatment.

13. The method of claim 7, wherein the trehalose is adsorbed to the zeolite.

14. The method of claim 7, wherein the zeolite is selected from the group consisting of FAU, BEA, DON, EMT, CFI, MOR, MAZ and OFF.

15. The method of claim 7, wherein the zeolite is selected from the group consisting of FAU, BEA, EMT, MOR, MAZ and OFF.

16. The method of claim 7, further comprising eluting the trehalose from the adsorbent.

17. The method of claim 7, wherein the solids are separated off from the fermentation broth by at least one method selected from the group consisting of filtration, pressure filtration, vacuum filtration, cake filtration, depth filtration, cross-flow filtration, microfiltration, sedimentation and centrifugation.

18. The method of claim 9, wherein said additional product is selected from the group consisting of organic acids, proteinogenic and nonproteinogenic amino acids, nucleotides, nucleosides, lipids, fatty acids, diols, carbohydrates, aromatic compounds, vitamins, co-factors, storage substances, polyhydroxyalkanoates, polyhydroxybutyrates, proteins, peptides and enzymes.

19. The method of claim 1, further comprising eluting the trehalose from the adsorbent.

20. The method of claim 1, wherein the zeolite is selected from the group consisting of FAU, BEA, EMT, MOR, MAZ and OFF.

21. The method of claim 1, further comprising purifying the trehalose by a method selected from the group consisting of activated carbon treatment, ultrafiltration, microfiltration, reverse osmosis and ion-exchange treatment.

22. A method for enriching trehalose from a fermentation broth, wherein the treholose is present in the fermentation broth at a concentration of less than 15 percent by weight measured on the dry weight of the fermentation broth, the method comprising separating off solids from the broth and exposing the broth to an adsorbent comprising a zeolite selected from the group consisting of FAU, BEA, EMT, MOR, MAZ and OFF, such that the trehalose is adsorbed to the zeolite.
